(19) United States
(12) Patent Application Publication
(10) Pub. No.: US 2025/0107760 A1
(43) Pub. Date: Apr. 3, 2025

(54) RADIOGRAPHY SYSTEM, AND OPERATION METHOD AND OPERATION PROGRAM THEREOF

(71) Applicant: FUJIFILM CORPORATION, Tokyo (JP)

(72) Inventors: Hisatsugu HORIUCHI, Kanagawa (JP); Kazuhiro MAKINO, Kanagawa (JP); Naoyuki NISHINO, Kanagawa (JP); Takeyasu KOBAYASHI, Kanagawa (JP)

(21) Appl. No.: 18/824,917

(22) Filed: Sep. 5, 2024

(30) Foreign Application Priority Data
Sep. 28, 2023 (JP) 2023-168775

(51) Int. Cl. A61B 6/10

(52) U.S. Cl. CPC A61B 6/105

(57) ABSTRACT

A radiography system includes a radiation detection device, a radiation source, a moving device capable of changing, in a plurality of directions, at least any of a translational position or a rotational position of the radiation source, the moving device including a plurality of detection sensors that detect positions of the radiation source in the plurality of directions, and a processor that controls the radiation source, in which the processor executes an interlock control for applying an interlock to prohibit the irradiation with the radiation by the radiation source in a case in which at least one of the positions in the plurality of directions is not included in an allowable range for a target position, and an assist control for giving a notification of or mechanically assisting in a direction in which the radiation source is to be moved in a case in which the interlock is applied.

### CROSS-REFERENCE TO RELATED APPLICATIONS

The present application claims priority under 35 U.S.C. § 119 to Japanese Patent Application No. 2023-168775, filed on Sep. 28, 2023. The above application is hereby expressly incorporated by reference, in its entirety, into the present application.

### BACKGROUND OF THE INVENTION

### 1. Technical Field

The technology of the present disclosure relates to a radiography system, and an operation method and an operation program thereof.

### 2. Description of the Related Art

In a radiography system, particularly in a case of performing fluoroscopy, it is necessary to accurately align a radiation source that emits radiation and a radiation detection device that detects the radiation transmitted through an examinee to generate a radiation image. In a case in which radiography is performed in a state in which the radiation source and the radiation detection device are not accurately aligned with each other, re-imaging is required, and the examinee is exposed to unnecessary radiation.

JP2008-506442A does not disclose fluoroscopy, but discloses that irradiation with radiation is permitted only in a case in which a radiation source and a radiation detection device are accurately aligned with each other (that is, an interlock control). In addition, JP2008-506442A discloses that, in a case in which a user aligns a radiation source with a radiation detection device, the proximity to a target position is indicated by color or voice.

### SUMMARY

According to the technology disclosed in JP2008-506442A, a user can align a radiation source while referring to color or voice to indicate the proximity to a target position. However, with the technology disclosed in JP2008-506442A, once positioning has been performed, the user cannot instantly ascertain the occurrence of misregistration, even in a case in which a misregistration occurs. In fluoroscopy, in a case in which a misregistration occurs, irradiation with radiation is prohibited. Therefore, the user needs to perform alignment again from the beginning, and there is a problem that alignment cannot be efficiently performed.

An object of the technology of the present disclosure is to provide a radiography system that enables efficient alignment of a radiation source, and an operation method and an operation program thereof.

In order to achieve the above object, according to an aspect of the present disclosure, there is provided a radiography system comprising a radiation detection device configured to detect radiation to generate a radiation image, a radiation source configured to irradiate the radiation detection device with the radiation, a moving device capable of changing, in a plurality of directions, at least any of a translational position or a rotational position of the radiation source with respect to the radiation detection device, the moving device including a plurality of detection sensors that detect positions of the radiation source in the plurality of directions, and a processor configured to control the radiation source, in which the processor is configured to execute an interlock control for applying an interlock to prohibit the irradiation with the radiation by the radiation source in a case in which, based on detection values from the plurality of detection sensors, at least one of the positions in the plurality of directions is not included in an allowable range for a target position, and an assist control for giving a notification of or mechanically assisting in a direction in which the radiation source is to be moved in a case in which the interlock is applied.

It is preferable that the processor is configured to determine which of a first imaging mode in which alignment of the radiation source with respect to the radiation detection device is prioritized and a second imaging mode in which quick imaging is prioritized over the alignment has been selected, and perform the interlock control and the assist control in a case in which it is determined that the first imaging mode has been selected.

It is preferable that the processor is configured to determine that the first imaging mode has been selected in a case in which a fluoroscopy switch for giving an instruction to execute fluoroscopy is pressed.

It is preferable that the processor is configured to determine that the first imaging mode has been selected in a case in which switching is made from a general imaging mode for executing general imaging to a fluoroscopy mode for executing fluoroscopy.

It is preferable that the processor is configured to determine that the first imaging mode has been selected in a case in which a general imaging switch for giving an instruction to execute general imaging is pressed in a fluoroscopy mode for executing fluoroscopy.

It is preferable that the radiography system further comprises a plurality of indicators corresponding to the plurality of directions, and the processor is configured to turn on an indicator corresponding to a direction having a position outside the allowable range among the plurality of indicators in the assist control.

It is preferable that the radiography system further comprises at least one brake mechanism configured to restrict movement of the radiation source in the plurality of directions, and an operating device that is provided with a plurality of brake release buttons for releasing a brake applied by the brake mechanism, and the plurality of indicators are provided in a vicinity of the plurality of brake release buttons.

It is preferable that the processor is configured to, in a case in which the indicator corresponding to the direction having the position outside the allowable range is turned on, change a lighting color in accordance with an amount of deviation between the position of the radiation source and the target position.

It is preferable that the processor is configured to, in a case in which the indicator corresponding to the direction having the position outside the allowable range is turned on, change a blinking cycle in accordance with an amount of deviation between the position of the radiation source and the target position.

It is preferable that the processor is configured to display, in the assist control, a device structure diagram that schematically shows the radiation source and the direction in which the radiation source is to be moved on a display.

It is preferable that the radiography system further comprises at least one brake mechanism configured to restrict movement of the radiation source in the plurality of directions, and an operating device that is provided with a plurality of brake release buttons for releasing a brake applied by the brake mechanism, and the processor is configured to allow only a brake corresponding to a direction having a position outside the allowable range to be released.

It is preferable that the radiography system further comprises at least one brake mechanism configured to restrict movement of the radiation source in the plurality of directions, and an operating device that is provided with a plurality of brake release buttons for releasing a brake applied by the brake mechanism, the plurality of brake release buttons include a simultaneous release button for simultaneously releasing brakes corresponding to two or more of the plurality of directions, and the processor is configured to, in a case in which the simultaneous release button is pressed, release only a brake corresponding to a direction having a position outside the allowable range.

It is preferable that the processor is configured to control the brake mechanism whose brake has been released, and apply a load in a direction opposite to a direction toward the target position.

It is preferable that the processor is configured to perform pulse duty driving on the brake mechanism whose brake has been released; and change a duty in accordance with a distance between the position of the radiation source and the target position.

It is preferable that the processor is configured to determine whether fluoroscopy intended by a user is upright fluoroscopy or decubitus fluoroscopy based on whether the positions in the plurality of directions are included more in an allowable range of a target position for upright fluoroscopy or an allowable range of a target position for decubitus fluoroscopy, and execute the assist control for a direction having a position outside the allowable range.

It is preferable that the radiography system further comprises a brake mechanism configured to restrict movement of the radiation source in a rotation direction among the plurality of directions, and an operating device that is provided with a brake release button for releasing a brake applied by the brake mechanism, and the brake mechanism includes a plunger for aligning the radiation source to a specific rotational position.

It is preferable that the processor is configured to cause the brake mechanism to apply the brake, after a delay time has elapsed from a point in time at which it is detected that pressing of the brake release button has been released.

It is preferable that the moving device includes a ceiling device or a floor device.

According to another aspect of the present disclosure, there is provided an operation method of a radiography system including a radiation detection device configured to detect radiation to generate a radiation image, a radiation source configured to irradiate the radiation detection device with the radiation, and a moving device capable of changing, in a plurality of directions, at least any of a translational position or a rotational position of the radiation source with respect to the radiation detection device, the moving device including a plurality of detection sensors that detect positions of the radiation source in the plurality of directions, the operation method comprising executing, by a processor, an interlock control for applying an interlock to prohibit the irradiation with the radiation by the radiation source in a case in which, based on detection values from the plurality of detection sensors, at least one of the positions in the plurality of directions is not included in an allowable range for a target position, and an assist control for giving a notification of or mechanically assisting in a direction in which the radiation source is to be moved in a case in which the interlock is applied.

According to still another aspect of the present disclosure, there is provided an operation program for operating a radiography system including a radiation detection device configured to detect radiation to generate a radiation image, a radiation source configured to irradiate the radiation detection device with the radiation, and a moving device capable of changing, in a plurality of directions, at least any of a translational position or a rotational position of the radiation source with respect to the radiation detection device, the moving device including a plurality of detection sensors that detect positions of the radiation source in the plurality of directions, the operation program causing a processor to execute an interlock control for applying an interlock to prohibit the irradiation with the radiation by the radiation source in a case in which, based on detection values from the plurality of detection sensors, at least one of the positions in the plurality of directions is not included in an allowable range for a target position, and an assist control for giving a notification of or mechanically assisting in a direction in which the radiation source is to be moved in a case in which the interlock is applied.

According to the technology of the present disclosure, it is possible to provide a radiography system that enables efficient alignment of a radiation source, and an operation method and an operation program thereof.

### BRIEF DESCRIPTION OF THE DRAWINGS

Exemplary embodiments according to the technique of the present disclosure will be described in detail based on the following figures, wherein:

FIG. 1 is a diagram schematically showing a configuration of a radiography system,

FIG. 2 is a perspective view showing a configuration of a radiation irradiation device,

FIG. 3 is a front view schematically showing a configuration of an operating device,

FIG. 4 is a block diagram showing an electric configuration of the radiography system,

FIG. 5 is a block diagram showing an example of a configuration of a controller,

FIG. 6 is a flowchart showing an example of an interlock control,

FIG. 7 is a flowchart showing an example of an irradiation control,

FIG. 8 is a flowchart showing an example of an assist control,

FIG. 9 is a diagram showing an example of a case in which a position of a radiation source is shifted in a Z-axis direction,

FIG. 10 is a diagram showing an example in which an indicator is turned on,

FIG. 11 is a diagram showing an example of displaying a device structure diagram on a display of a console,

FIG. 12 is a diagram showing an example of displaying a device structure diagram on a display of an operating device,

FIG. 13 is a flowchart showing an example of an assist control according to a second embodiment,

FIG. 14 is a flowchart showing an assist control according to a modification example of the second embodiment,

FIG. 15 is a diagram showing an example in which a brake is released only in a left direction toward a target position,

FIG. 16 is a diagram showing an example of a configuration of a brake mechanism,

FIG. 17 is a diagram illustrating pulse duty driving,

FIG. 18 is a diagram showing an example of a screen for general imaging,

FIG. 19 is a diagram showing an example of a target position for upright fluoroscopy and a target position for decubitus fluoroscopy,

FIG. 20 is a flowchart showing an assist control according to a modification example.

FIG. 21 is a side view of the radiation irradiation device,

FIG. 22 is a front view of the brake mechanism,

FIG. 23 is a diagram illustrating an alignment function of a plunger,

FIG. 24 is a diagram illustrating a delay time for pressing a brake release button,

FIG. 25 is a diagram showing an example of a floor device, and

FIG. 26 is a diagram illustrating a quick imaging priority mode and an alignment priority mode.

### DETAILED DESCRIPTION

An example of an embodiment according to the technology of the present disclosure will be described with reference to the accompanying drawings.

### First Embodiment

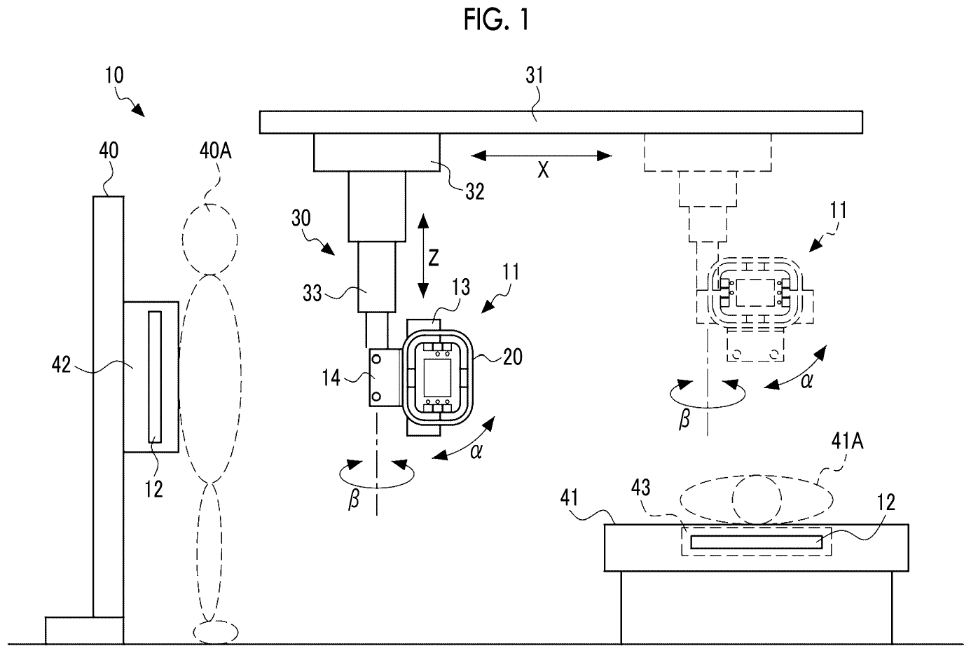

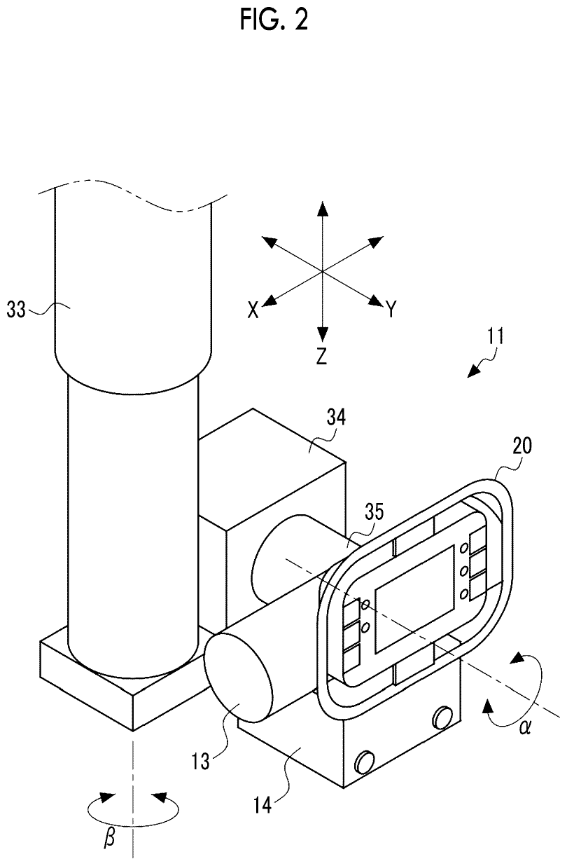

FIG. 1 schematically shows a configuration of a radiography system 10. FIG. 2 shows a configuration of a radiation irradiation device 11. The radiography system 10 includes the radiation irradiation device 11 and a radiation detection device 12. The radiography system 10 can perform both upright imaging and decubitus imaging.

The radiation irradiation device 11 includes a radiation source 13 that generates radiation (for example, X-rays) and a collimator device 14 that has a movable stop and limits an irradiation range of the radiation. The radiation source 13 is also called a radiation tube. The radiation irradiation device 11 also includes an operating device 20 that is operated by a user and a moving device 30 for moving the radiation source 13.

In the present disclosure, a position includes a position in a translational direction along a certain axis (that is, a translational position) and a position in a rotation direction about a certain axis (that is, a rotational position). Additionally, in the present disclosure, a movement includes changing a translational position (that is, moving in a linear direction) and changing a rotational position (that is, changing an angle). That is, the moving device 30 can change at least any of the translational position or the rotational position of the radiation source 13 in a plurality of directions.

In the imaging room, an upright imaging table 40 used in a case in which radiography in an upright posture is performed and a decubitus imaging table 41 used in a case in which radiography in a decubitus posture is performed are installed. A space on the front side of the upright imaging table 40 is an imaging position 40A of an examinee in a case in which radiography is performed in the upright posture. A space on an upper side of the decubitus imaging table 41 is an imaging position 41A of the examinee in a case in which the radiography is performed in the decubitus posture.

The radiation detection device 12 is a portable type and can be attached to and detached from the upright imaging table 40 and the decubitus imaging table 41. For example, the radiation detection device 12 is an electronic cassette incorporating a flat panel detector (FPD). The upright imaging table 40 is provided with a holder 42 for mounting the radiation detection device 12 thereon. In a case in which a radiation image is captured in an upright posture, the radiation detection device 12 is mounted on the holder 42 of the upright imaging table 40. In a case in which a radiation image is captured in a decubitus posture, the radiation detection device 12 is mounted on a holder 43 of the decubitus imaging table 41.

The radiation detection device 12 absorbs radiation that has transmitted through a part of the examinee to be imaged, generates electric charges, and generates a radiation image based on the generated electric charges.

The moving device 30 holds the position of the radiation source 13 to be changeable in an X-axis direction, a Y-axis direction, and a Z-axis direction. Here, the X-axis direction, the Y-axis direction, and the Z-axis direction are directions orthogonal to each other, and the Z-axis direction is a vertical direction. In addition, the moving device 30 holds the position of the radiation source 13 to be changeable in an α direction and a β direction. Here, the β direction is a rotation direction with the Z-axis as a rotation center. The α direction is a rotation direction with an axis extending in a horizontal direction (depending on the rotational position in the β direction) as a rotation center.

The moving device 30 includes a rail 31, a traveling vehicle 32, a suspension frame 33, a radiation source holding portion 34, and a rotation support portion 35. The traveling vehicle 32 is held by the rail 31 to be movable in the X-axis direction and the Y-axis direction. For example, the rail 31 is composed of a fixed rail that extends in the Y-axis direction and is laid and disposed on the ceiling and a movable rail that extends in the X-axis direction and is provided to be movable along the fixed rail. The traveling vehicle 32 is held on the movable rail to be freely movable.

The suspension frame 33 is disposed to be extendable and contractible in the Z direction, and an upper end thereof is fixed to the traveling vehicle 32. The radiation source holding portion 34 is held at a lower end of the suspension frame 33 to be rotatable in the β direction with the Z-axis as a rotation center. The radiation source 13 is held by the radiation source holding portion 34 via the rotation support portion 35. The radiation source 13 is supported by the rotation support portion 35 to be rotatable in the α direction. The collimator device 14 and the operating device 20 are fixed to the radiation source 13.

The traveling vehicle 32, the suspension frame 33, and the like may be configured to be electrically driven by a driving unit such as a motor. Hereinafter, the rail 31, the traveling vehicle 32, and the suspension frame 33 may be referred to as a ceiling device.

The user can change the position of the radiation source 13 by applying a force to the operating device 20 in a desired direction. By changing the position of the radiation source 13, the irradiation position or irradiation angle of the radiation changes.

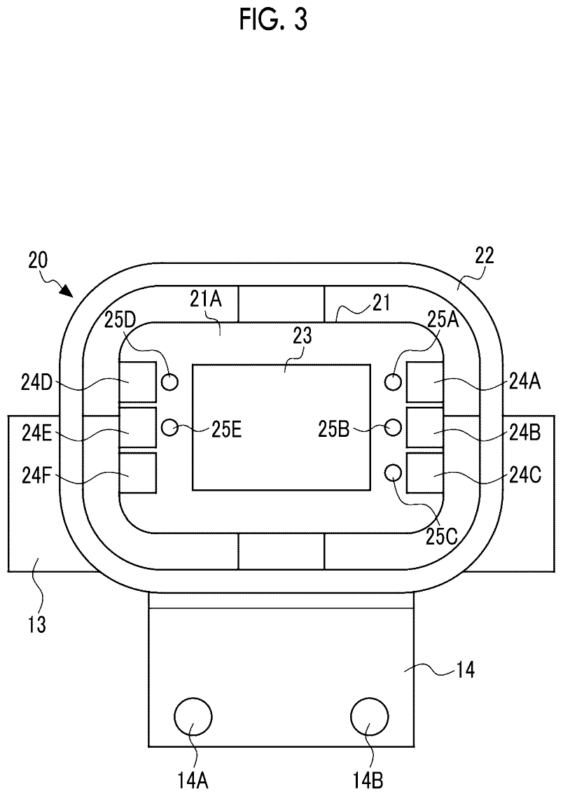

FIG. 3 schematically shows a configuration of the operating device 20. The operating device 20 includes a device main body 21 and a grip portion 22 fixed to the device main body 21. The device main body 21 is fixed to the radiation source 13. The grip portion 22 is disposed around the device main body 21 and is gripped by the user.

A display 23 for displaying various types of information is provided at the center of a front surface 21A of the device main body 21. For example, the display 23 is a display device such as a liquid crystal display.

Additionally, brake release buttons 24A to 24F are provided on the sides of the front surface 21A. Hereinafter, in a case in which it is not necessary to distinguish between the brake release buttons 24A to 24E, the brake release buttons 24A to 24E will simply be referred to as brake release buttons 24. The brake release buttons 24A to 24F are an example of a “plurality of brake release buttons” according to the technology of the present disclosure.

The brake release buttons 24A to 24F are operation buttons for releasing the brake applied to the radiation source 13 by a brake mechanism 36, which will be described later. The brake restricts the movement of the radiation source 13 by the moving device 30. While any of the brake release buttons 24A to 24F is being pressed, the brake applied by the brake mechanism 36 corresponding to the pressed brake release button 24 is released.

The brake release button 24A is an operation button for releasing the brake on the radiation source 13 in the X-axis direction. The brake release button 24B is an operation button for releasing the brake on the radiation source 13 in the Y-axis direction. The brake release button 24C is an operation button for releasing the brake on the radiation source 13 in the Z-axis direction. The brake release button 24D is an operation button for releasing the brake on the radiation source 13 in the α direction. The brake release button 24E is an operation button for releasing the brake on the radiation source 13 in the β direction. The brake release button 24F is an operation button for simultaneously releasing three brakes on the radiation source 13 in the X-axis direction, the Y-axis direction, and the Z-axis direction. Hereinafter, the brake release button 24F may also be referred to as a “simultaneous release button 24F”. It is sufficient that the simultaneous release button 24F simultaneously releases two or more brakes.

Indicators 25A to 25E are provided in the vicinity of the brake release buttons 24A to 24E, respectively. The indicators 25A to 25E are turned on in a case in which the alignment of the radiation source 13 with respect to the radiation detection device 12 is necessary and the position of the radiation source 13 is outside an allowable range. The indicators 25A to 25E are, for example, light emitting diodes (LEDs). Hereinafter, in a case in which it is not necessary to distinguish between the indicators 25A to 25E, the indicators 25A to 25E will simply be referred to as indicators 25. The indicators 25A to 25E are an example of a “plurality of indicators” according to the technology of the present disclosure.

The indicator 25A is turned on in a case in which the position of the radiation source 13 in the X-axis direction is outside the allowable range. The indicator 25B is turned on in a case in which the position of the radiation source 13 in the Y-axis direction is outside the allowable range. The indicator 25C is turned on in a case in which the position of the radiation source 13 in the Z-axis direction is outside the allowable range. The indicator 25D is turned on in a case in which the position of the radiation source 13 in the α direction is outside the allowable range. The indicator 25E is turned on in a case in which the position of the radiation source 13 in the B direction is outside the allowable range.

The collimator device 14 is also provided with adjustment knobs 14A and 14B for adjusting an irradiation field of the radiation. By operating the adjustment knobs 14A and 14B, the size of the irradiation field can be changed in two directions orthogonal to the optical axis of the radiation. For example, by operating the adjustment knob 14A, the size of the irradiation field can be changed in a first direction orthogonal to the optical axis of the radiation. By operating the adjustment knob 14B, the size of the irradiation field can be changed in a second direction that is orthogonal to the optical axis of the radiation and orthogonal to the first direction.

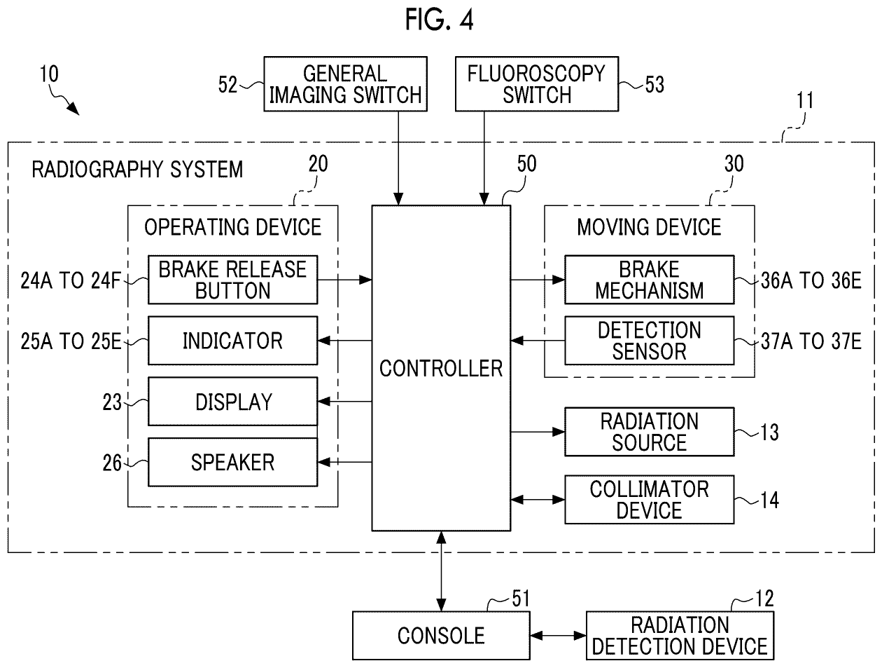

FIG. 4 shows an electric configuration of the radiography system 10. The radiation irradiation device 11 includes a controller 50 that controls each unit of the radiation irradiation device 11, in addition to the radiation source 13, the collimator device 14, the operating device 20, and the moving device 30. A console 51 serving as a control device is connected to the controller 50.

The console 51 is connected to the radiation detection device 12 in a wired or wireless manner. The console 51 transmits a synchronization signal to the radiation irradiation device 11 and the radiation detection device 12, transmits exposure conditions to the radiation irradiation device 11, receives and displays a radiation image from the radiation detection device 12, and the like. Furthermore, the console 51 enables a selection operation of an imaging menu by the user, a switching operation of an imaging mode, and the like. The imaging modes include a general imaging mode and a fluoroscopy mode.

A general imaging switch 52 and a fluoroscopy switch 53 are connected to the controller 50. The general imaging switch 52 is an irradiation start switch that is operated by the user to give an instruction to execute general imaging in the general imaging mode. The general imaging refers to performing one time of radiography in response to the operation of the general imaging switch 52. For example, the general imaging switch 52 is a two-step motion switch. In a case in which the user presses the general imaging switch 52 to the first position, an anode (not shown) included in the radiation source 13 starts to rotate. Then, in a case in which the user presses the general imaging switch 52 to the second position, a tube voltage is applied between the anode and the cathode from a tube voltage generator (not shown), and therefore radiation is generated and emitted to the radiation detection device 12.

The fluoroscopy switch 53 is an irradiation start switch that is operated by the user to give an instruction to execute fluoroscopy in the fluoroscopy mode. The fluoroscopy refers to continuous radiography while the fluoroscopy switch 53 is being operated. For example, the fluoroscopy switch 53 is a foot switch that is pressed by the user with his/her foot. While the user is pressing the fluoroscopy switch 53, the radiation source 13 generates continuous or periodic pulsed radiation and the pulsed radiation is emitted to the radiation detection device 12.

For example, the user can operate the console 51 to designate continuous irradiation or pulse irradiation as a radiation irradiation method of the fluoroscopy. The continuous irradiation is an irradiation method in which radiation is continuously emitted from the radiation source 13 during the fluoroscopy. The pulse irradiation is an irradiation method in which radiation is emitted in a pulsed manner from the radiation source 13 in synchronization with the frame rate of imaging during fluoroscopy.

The operating device 20 is provided with a speaker 26 for outputting a voice in addition to the brake release buttons 24A to 24E, the indicators 25A to 25E, and the display 23.

The moving device 30 includes brake mechanisms 36A to 36E and detection sensors 37A to 37E, in addition to the above-mentioned mechanism for moving the radiation source 13. The brake mechanisms 36A to 36E are each composed of, for example, an electromagnetic brake, and the operation thereof is controlled by the controller 50. The detection sensors 37A to 37E are composed of potentiometers, microswitches, photosensors, and the like, and output position detection values to the controller 50. Hereinafter, in a case in which it is not necessary to distinguish between the brake mechanisms 36A to 36E, the brake mechanisms 36A to 36E will simply be referred to as brake mechanisms 36. The brake mechanisms 36A to 36E are an example of a “plurality of brake mechanisms” according to the technology of the present disclosure. The detection sensors 37A to 37E are an example of a “plurality of detection sensors” according to the technology of the present disclosure.

The brake mechanism 36A is a mechanism that restricts the movement of the radiation source 13 in the X-axis direction, and is provided, for example, between the rail 31 and the traveling vehicle 32. The brake mechanism 36B is a mechanism that restricts the movement of the radiation source 13 in the Y-axis direction, and is provided, for example, on the rail 31. The brake mechanism 36C is a mechanism that restricts the movement of the radiation source 13 in the Z-axis direction, and is provided, for example, on the suspension frame 33. The brake mechanism 36D is a mechanism that restricts the movement (that is, the rotation) of the radiation source 13 in the α direction, and is provided, for example, between the radiation source holding portion 34 and the rotation support portion 35. The brake mechanism 36E is a mechanism that restricts the movement (that is, the rotation) of the radiation source 13 in the β direction, and is provided, for example, between the radiation source holding portion 34 and the rotation support portion 35.

The detection sensor 37A detects a position of the radiation source 13 in the X-axis direction. The detection sensor 37B detects a position of the radiation source 13 in the Y-axis direction. The detection sensor 37C detects a position of the radiation source 13 in the Z-axis direction. The detection sensor 37D detects a position of the radiation source 13 in the a direction (that is, the rotational position). The detection sensor 37E detects a position of the radiation source 13 in the β direction (that is, the rotational position).

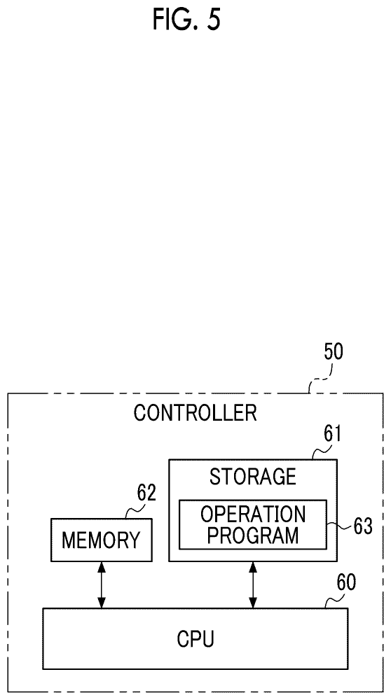

FIG. 5 shows an example of the configuration of the controller 50. The controller 50 is composed of, for example, a central processing unit (CPU) 60, a storage 61, and a memory 62. The storage 61 stores an operation program 63 and various types of data. The storage 61 is a non-volatile storage device such as a flash memory. The memory 62 is a volatile storage device, such as a random-access memory (RAM) and is used as a work memory. The CPU 60 operates each unit based on the operation program 63 to implement various functions. The CPU 60 is an example of a “processor” according to the technology of the present disclosure.

The configuration of the controller included in the console 51 is similar to the configuration of the controller 50. The controller of the console 51 is composed of, for example, a CPU, a storage that stores an operation program, and a memory. The console 51 also comprises an operation unit such as a keyboard and a display such as a display.

In the fluoroscopy mode, the controller 50 executes an interlock control, an irradiation control, and an assist control. The interlock control is control for applying an interlock to prohibit irradiation with radiation in a case in which at least one of the positions of the radiation source 13 in a plurality of directions is outside the allowable range in the fluoroscopy mode. This is because, in the fluoroscopy, it is required to accurately align the radiation source 13 with the radiation detection device 12 in order to prevent re-imaging. The irradiation control is control for causing the radiation source 13 to generate radiation in response to the operation of the fluoroscopy switch 53. The assist control is control for giving a notification of or mechanically assisting in a direction in which the radiation source 13 should be moved in a case in which the interlock is applied.

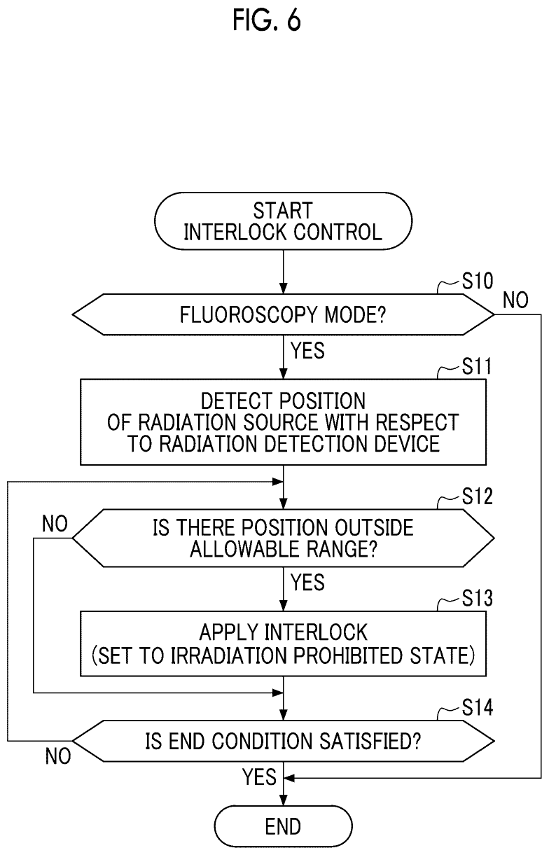

FIG. 6 shows an example of an interlock control. In the interlock control, first, the controller 50 determines whether or not the imaging mode is the fluoroscopy mode (Step S10). In a case in which the controller 50 determines that the imaging mode is not the fluoroscopy mode (Step S10: NO), the controller 50 ends the process.

In a case in which the controller 50 determines that the imaging mode is the fluoroscopy mode (Step S10: YES), the controller 50 detects the position of the radiation source 13 with respect to the radiation detection device 12 based on the position detection values input from the detection sensors 37A to 37E (Step S11). Specifically, the positions in the X-axis direction, the Y-axis direction, the Z-axis direction, the α direction, and the β direction are detected.

Next, the controller 50 determines whether or not there is a position outside an allowable range in the plurality of detected positions in executing the fluoroscopy (Step S12). The fluoroscopy includes upright fluoroscopy performed using the upright imaging table 40 and decubitus fluoroscopy performed using the decubitus imaging table 41. The target position of the radiation source 13 with respect to the radiation detection device 12 and the allowable range for the target position are different between the upright fluoroscopy and the decubitus fluoroscopy. The target positions and allowable ranges in the cases of upright fluoroscopy and decubitus fluoroscopy are stored as data in the memory 62. For example, in the case of the upright fluoroscopy, the target position in the X-axis direction is a source-to-image distance (SID) (for example, 1200 mm), the target position in the Y direction is 0 mm, the target position in the Z direction is a center position of an image receiving surface, the target position in the a direction is 90°, and the target position in the β direction is 0°. The allowable range is a range centered around the set value. The allowable range may be set to a different value for each of the target positions in the X-axis direction, the Y-axis direction, the Z-axis direction, the a direction, and the β direction.

In a case in which there is a position outside the allowable range (Step S12: YES), the controller 50 applies an interlock and prohibits irradiation with radiation from the radiation source 13 (Step S13), and transitions the process to Step S14. On the other hand, in a case in which there is no position outside the allowable range (Step S12: NO), the controller 50 transitions the process to Step S14 without applying an interlock. In Step S14, the controller 50 determines whether or not an end condition is satisfied (Step S14). In a case in which the end condition is not satisfied (Step S14: NO), the controller 50 returns the process to Step S12. In a case in which the end condition is satisfied (Step S14: YES), the controller 50 ends the process. For example, the end condition is that the console 51 receives an end instruction issued by the user.

The controller 50 may align the radiation source 13 while the user is pressing the brake release button 24, and perform the above-described interlock control in a case in which the user releases the pressing of the brake release button 24.

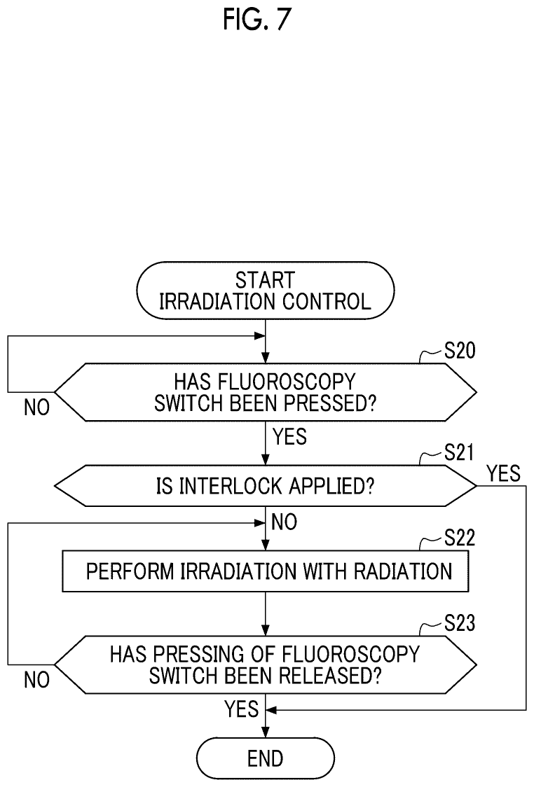

FIG. 7 shows an example of an irradiation control. In the irradiation control, first, the controller 50 determines whether or not the fluoroscopy switch 53 has been pressed (Step S20). In a case in which it is determined that the fluoroscopy switch 53 has been pressed (Step S20: YES), the controller 50 determines whether or not the interlock is applied (Step S21). In a case in which it is determined that the interlock is not applied (Step S21: NO), the controller 50 causes the radiation source 13 to perform irradiation with radiation (Step S22). On the other hand, in a case in which it is determined that the interlock is applied (Step S21: YES), the controller 50 ends the process without causing the radiation source 13 to perform the irradiation with radiation.

The controller 50 determines whether or not the pressing of the fluoroscopy switch 53 has been released after the radiation source 13 is caused to perform the irradiation with radiation (Step S23). In a case in which it is determined that the pressing of the fluoroscopy switch 53 has not been released (Step S23: NO), the controller 50 transitions the process to Step S22 and causes the radiation source 13 to continue the irradiation with radiation. On the other hand, in a case in which it is determined that the pressing of the fluoroscopy switch 53 has been released (Step S23: YES), the controller 50 causes the radiation source 13 to end the irradiation with radiation, and ends the process.

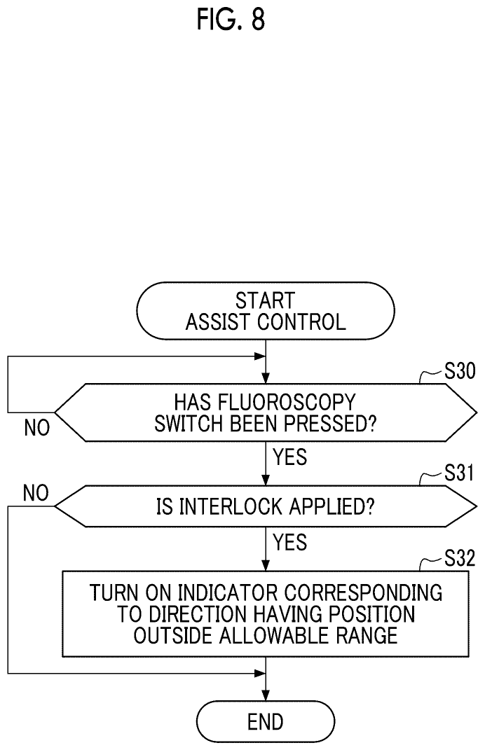

FIG. 8 shows an example of an assist control. In the assist control, first, the controller 50 determines whether or not the fluoroscopy switch 53 has been pressed (Step S30). In a case in which it is determined that the fluoroscopy switch 53 has been pressed (Step S30: YES), the controller 50 determines whether or not the interlock is applied (Step S31). In a case in which it is determined that the interlock is not applied (Step S31: NO), the controller 50 ends the process.

On the other hand, in a case in which it is determined that the interlock is applied (Step S31: YES), the controller 50 turns on the indicator 25 corresponding to the direction having a position outside the allowable range among the indicators 25A to 25E based on the position information of the radiation source 13 detected by the interlock control (Step S32).

In the present embodiment, the controller 50 determines whether or not the assist control is required based on whether or not the fluoroscopy switch 53 has been pressed.

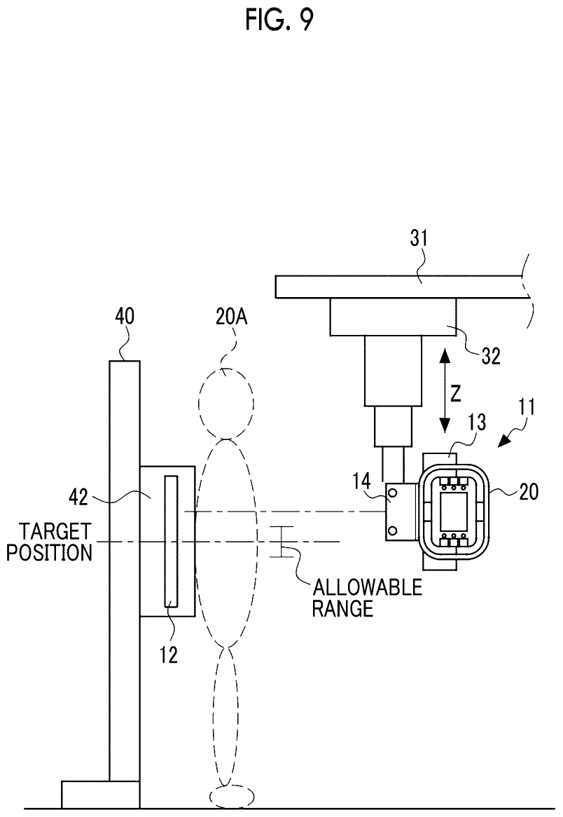

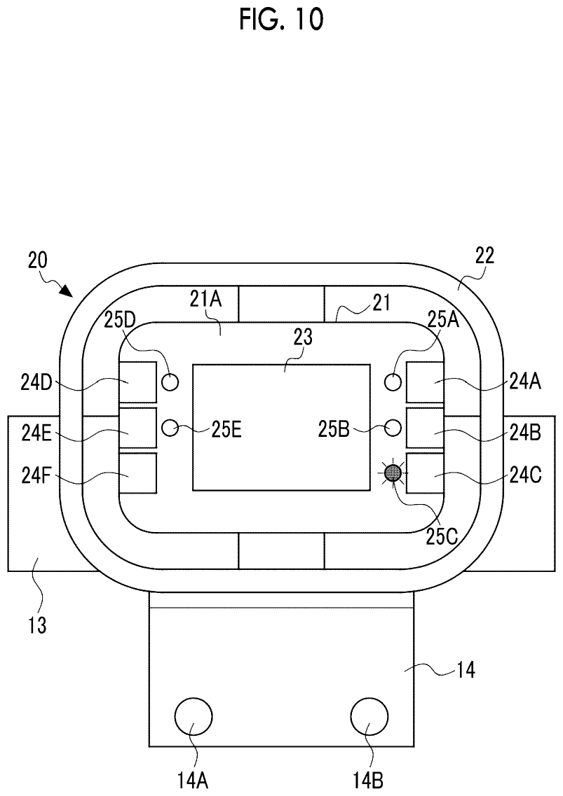

FIG. 9 shows an example in which the position of the radiation source 13 is shifted in the Z-axis direction. As shown in FIG. 9, in a case in which the position of the radiation source 13 in the Z direction is outside the allowable range with the center of the image receiving surface of the radiation detection device 12 as a target position, as shown in FIG. 10, the controller 50 turns on the indicator 25C corresponding to the brake release button 24C.

The user can ascertain from the fact that the indicator 25C is turned on that the position of the radiation source 13 has shifted in the Z direction. Accordingly, the user can align the radiation source 13 in the Z-axis direction by pressing the brake release button 24C and applying a force to the operating device 20. The same applies to a case in which other positions are outside the allowable range.

As described above, according to the present embodiment, in a case in which the fluoroscopy switch 53 is pressed and the interlock is applied, the indicator 25 corresponding to the direction having a position outside the allowable range among the positions in the plurality of directions of the radiation source 13 is turned on. This enables the user to efficiently align the radiation source 13.

Various modification examples of the first embodiment will be described below.

In the above embodiment, the controller 50 turns on the indicator 25 corresponding to the direction having a position outside the allowable range, but the controller 50 may change the lighting color of the indicator 25 in accordance with the amount of deviation between the position of the radiation source 13 and the target position. For example, in a case in which the amount of deviation is a certain value or more, the controller 50 sets the lighting color of the indicator 25 to red, and in a case in which the amount of deviation is less than the certain value, the controller 50 sets the lighting color to yellow. Accordingly, the user can ascertain the magnitude of the amount of deviation.

In addition, the controller 50 may blink the indicator 25 corresponding to the direction having a position outside the allowable range blink. In this case, the controller 50 may change the blinking cycle of the indicator in accordance with the amount of deviation between the position of the radiation source 13 and the target position. For example, the controller 50 reduces the blinking cycle as the amount of deviation increases. Accordingly, the user can more accurately ascertain the magnitude of the amount of deviation.

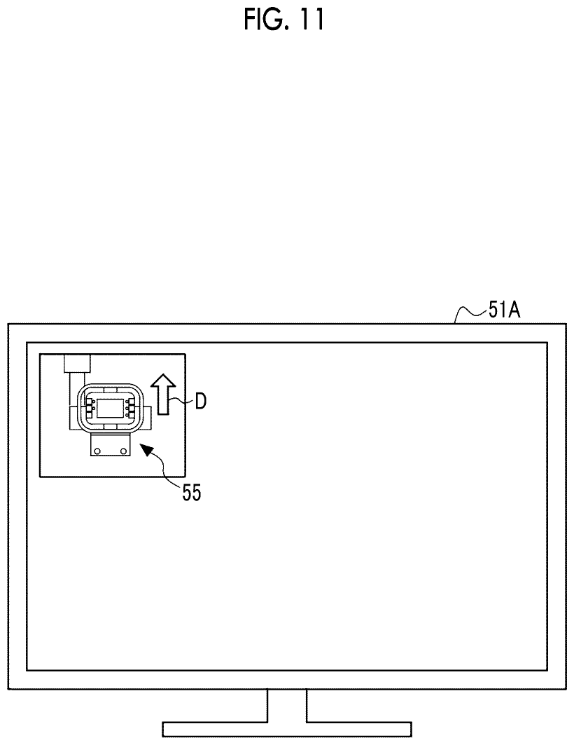

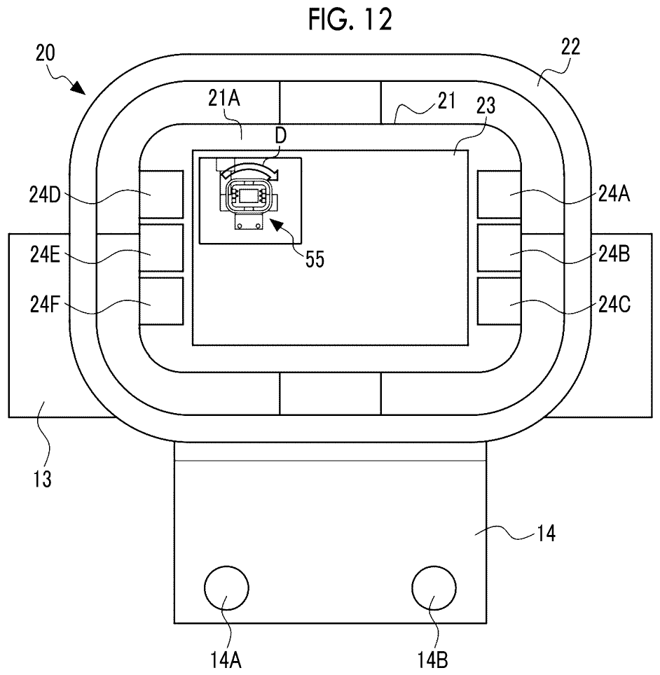

In the above embodiment, the controller 50 assists the user in alignment by turning on the indicator 25, but may also assist the user in alignment by displaying assist information on the display of the console 15 or the display 23 of the operating device 20. For example, as shown in FIG. 11, a device structure diagram 55 that schematically shows the radiation source 13 may be displayed on a part of a display 51A of the console 15, and an arrow D indicating a direction to be aligned may be displayed in the vicinity of the device structure diagram 55. FIG. 11 shows that the position of the radiation source 13 in the Z direction is outside the allowable range and should be aligned in the Z direction. In addition, as shown in FIG. 12, the device structure diagram 55 may be displayed on a part of the display 23 of the operating device 20, and an arrow D indicating a direction to be aligned may be displayed in the vicinity of the device structure diagram 55. FIG. 12 shows that the position of the radiation source 13 in the a direction is outside the allowable range and should be aligned in the α direction. In this case, the indicators 25A to 25E may not be provided.

In addition, also in the examples shown in FIGS. 11 and 12, the display form may be changed in accordance with the amount of deviation between the position of the radiation source 13 and the target position. For example, the color of the arrow D may be changed in accordance with the amount of deviation. In addition, the color of the arrow D may be made to blink, and the blinking cycle may be changed in accordance with the amount of deviation.

In addition, the controller 50 may also assist the user in alignment by controlling the speaker 26 to output a voice. For example, the direction to be aligned may be assisted by outputting a voice. In the present disclosure, a notification refers to conveying information to a user. The notification includes not only displaying information to the user, but also outputting a voice to the user.

### Second Embodiment

Next, a second embodiment will be described. In the above embodiment and each of the modification examples, the user is notified of the direction having a position outside the allowable range, and thus the alignment is assisted. In the present embodiment, the alignment is assisted by mechanically conveying to the user the direction having a position outside the allowable range.

The configuration of a radiography system according to the second embodiment is similar to the configuration of the radiography system 10 according to the first embodiment. In the present embodiment, the operating device 20 may not be provided with the indicators 25A to 25E. In the present embodiment, only the assist control by the controller 50 differs from the above embodiment. In the present embodiment, the controller 50 performs the assist control by controlling the brake mechanisms 36A to 36E instead of the indicators 25A to 25E.

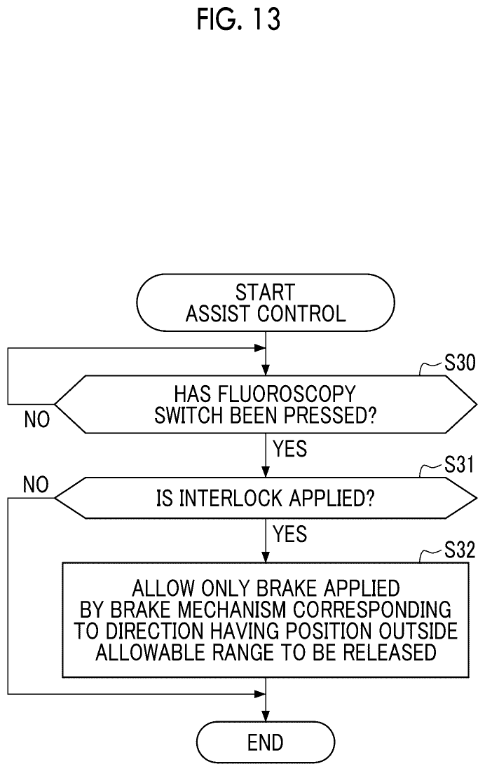

FIG. 13 shows an example of an assist control according to the second embodiment. The assist control according to the present embodiment differs from the assist control according to the first embodiment shown in FIG. 8 in only Step S32. In the present embodiment, in a case in which it is determined that the interlock is applied (Step S31: YES), the controller 50 allows only the brake applied by the brake mechanism 36 corresponding to the direction having a position outside the allowable range, among the brake mechanisms 36A to 36E, to be released, based on the position information of the radiation source 13 detected by the interlock control (Step S32). Accordingly, the user can ascertain the direction having a position outside the allowable range.

For example, as shown in FIG. 9, in a case in which the position of the radiation source 13 in the Z direction is outside the allowable range with the center of the image receiving surface of the radiation detection device 12 as a target position, the controller 50 allows only the brake applied by the brake mechanism 36C, among the brake mechanisms 36A to 36E, to be released. That is, the user can release the brake only in a case in which the brake release button 24C among the brake release buttons 24A to 24E is pressed, and can align the radiation source 13 in the Z direction while pressing the brake release button 24C.

As described above, according to the present embodiment, in a case in which the fluoroscopy switch 53 is pressed and the interlock is applied, only the brake applied by the brake mechanism 36 corresponding to the direction having a position outside the allowable range among the positions in the plurality of directions of the radiation source 13 can be released. This enables the user to efficiently align the radiation source 13.

Various modification examples of the second embodiment will be described below.

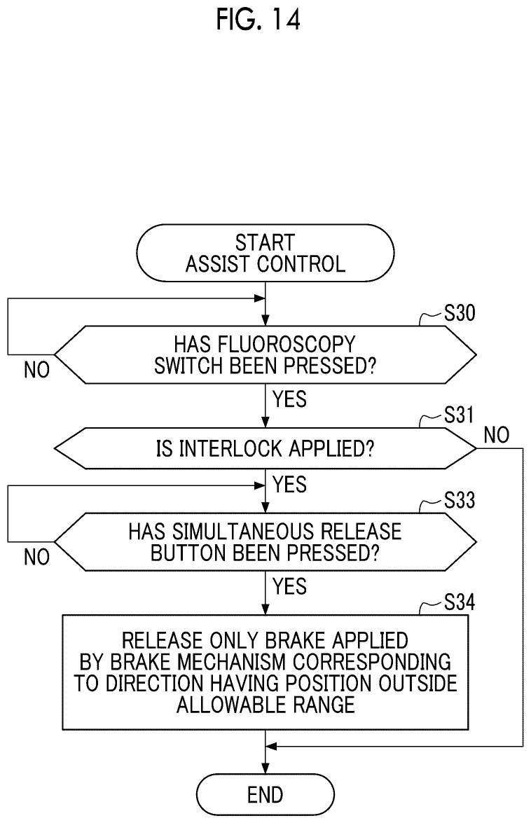

FIG. 14 shows an assist control according to a modification example of the second embodiment. The assist control according to the present modification example differs from the assist control according to the second embodiment only in that steps S33 and S34 are executed instead of Step S32. In the present modification example, in a case in which it is determined that the interlock is applied (Step S31: YES), the controller 50 determines whether or not the simultaneous release button 24F has been pressed (Step S33). In a case in which it is determined that the simultaneous release button 24F has been pressed (Step S33: YES), the controller 50 releases only the brake applied by the brake mechanism 36 corresponding to the direction having a position outside the allowable range among the X-axis direction, the Y-axis direction, and the Z-axis direction (Step S34). Accordingly, the user can ascertain the direction having a position outside the allowable range.

For example, as shown in FIG. 9, in a case in which the position of the radiation source 13 in the Z direction is outside the allowable range with the center of the image receiving surface of the radiation detection device 12 as a target position, the controller 50 releases only the brake applied by the brake mechanism 36C in a case in which the simultaneous release button 24F has been pressed. That is, the user can align the radiation source 13 in only the Z direction while pressing the simultaneous release button 24F.

In addition, in a case in which the brake mechanisms 36A to 36C are simultaneously pressed instead of the simultaneous release button 24F, the controller 50 may release only the brake applied by the brake mechanism 36 corresponding to the direction having a position outside the allowable range among the X-axis direction, the Y-axis direction, and the Z-axis direction.

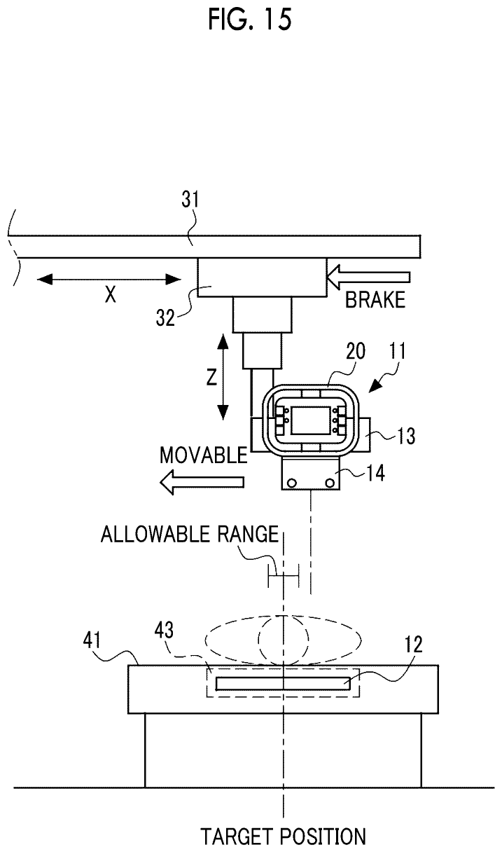

In addition, the controller 50 may release the brake applied by the brake mechanism 36 corresponding to the direction having a position outside the allowable range in only the direction toward the target position. For example, as shown in FIG. 15, in a case in which the position of the radiation source 13 in the X direction is outside the allowable range, the controller 50 may control the brake mechanism 36A such that the brake is released only in the left direction toward the target position and the brake is applied to the right side. In this case, the brake may not be a brake that completely restricts the movement of the radiation source 13. For example, the brake mechanism 36A may be controlled to apply a load in a direction opposite to a direction toward the target position by pulse duty driving, which will be described later. Accordingly, the user can easily ascertain the direction toward the target position and can perform the alignment more efficiently.

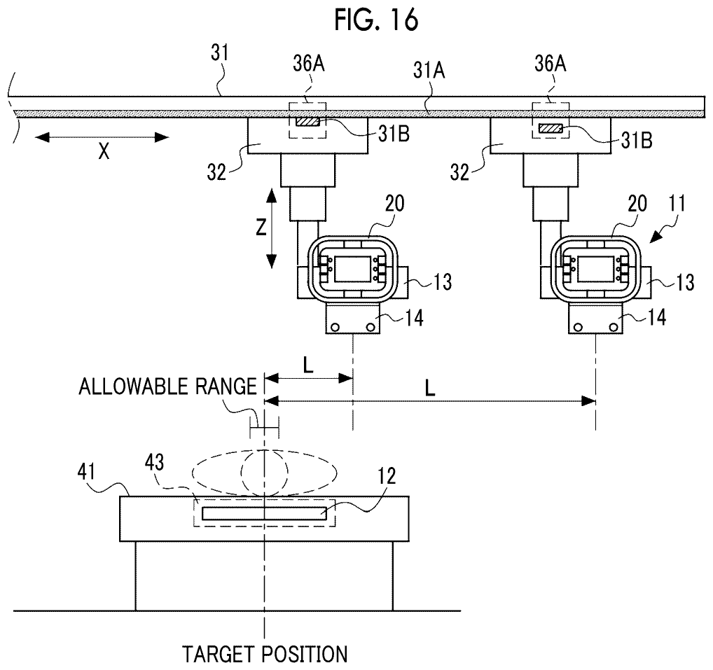

In addition, the controller 50 may change the load of the brake in accordance with the position of the radiation source 13 and the distance to the target position. As shown in FIG. 16, the brake mechanism 36A includes a brake pad 31A and an electromagnet 31B that are provided on the rail 31. The controller 50 performs on/off control of the brake mechanism 36A by controlling the current flowing through the electromagnet 31B. The controller 50 detects the position of the radiation source 13 and a distance L to the target position, and reduces the load as the distance L increases.

For example, the controller 50 performs pulse duty driving shown in FIG. 17. The pulse duty driving is a driving method in which the on and off of the brake are periodically repeated and the load can be changed by changing the duty. In a case in which the on and off cycle is denoted by T and the on period is denoted by H, the duty is expressed as H/T×100. The controller 50 reduces the duty as the distance L increases. The pulse duty driving is not limited to the X-axis direction, and can be applied to any of the Y-axis direction, the Z-axis direction, the α direction, and the β direction.

In this way, by reducing the load as the distance L increases, the user can easily move the radiation source 13 in a case in which the distance L is large, but it is difficult to move the radiation source 13 as the distance L decreases. Accordingly, it is possible to move the radiation source 13 to the vicinity of the target position at a high speed and to accurately align the radiation source 13 in the vicinity of the target position.

The control of the brake mechanism 36 described with reference to FIGS. 15 to 17 can also be applied in a case in which the user presses the brake release button 24 to move the radiation source 13 in accordance with the assist control according to the first embodiment or each of the modification examples.

### Other Modification Examples

A modification example common to the first and second embodiments will be described below.

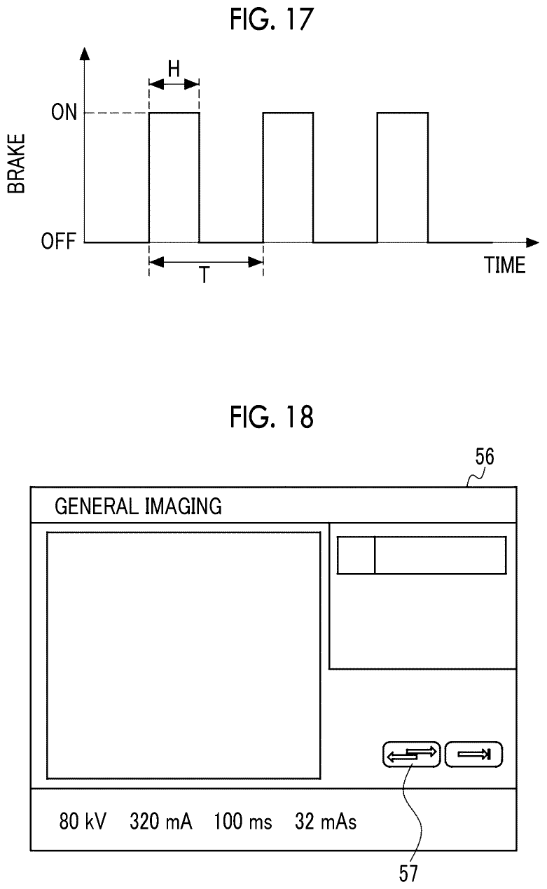

In each of the above embodiments, the controller 50 determines whether or not the assist control is required based on whether or not the fluoroscopy switch 53 has been pressed, but may determine whether or not the assist control is required based on whether or not the imaging mode has been switched to the fluoroscopy mode. For example, as shown in FIG. 18, the controller 50 may perform the above-mentioned assist control by determining that the assist control is required in a case in which the imaging mode is switched to the fluoroscopy mode from the general imaging mode by pressing a mode switching button 57 in a general imaging screen 56 displayed on the display 51A of the console 15 or the like.

In addition, in the settings of the imaging menu in the console 15, it is often not possible to set whether the fluoroscopy is upright fluoroscopy or decubitus fluoroscopy. Therefore, the controller 50 may determine whether the fluoroscopy intended by the user is the upright fluoroscopy or the decubitus fluoroscopy based on whether positions of the radiation source 13 in the plurality of directions are included more in an allowable range of a target position for upright fluoroscopy or an allowable range of a target position for decubitus fluoroscopy.

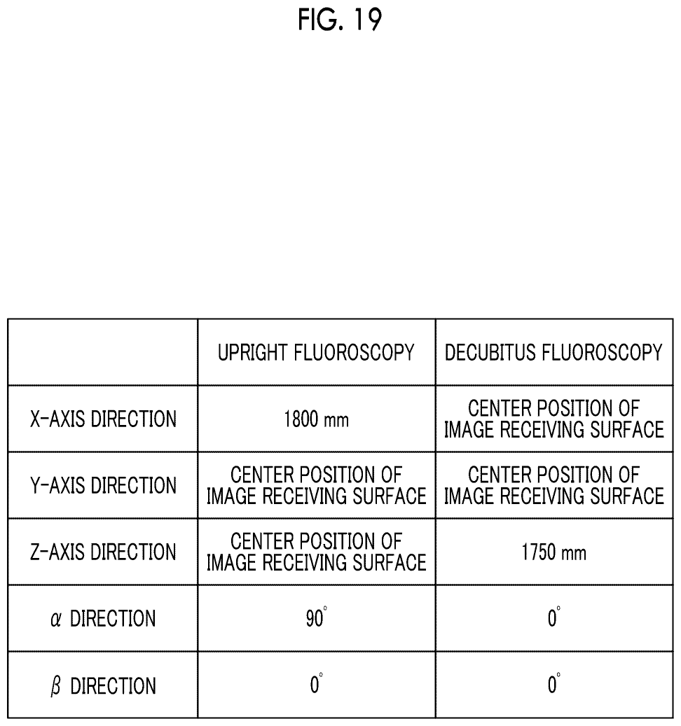

FIG. 19 shows an example of a target position for upright fluoroscopy and a target position for decubitus fluoroscopy. As shown in FIG. 19, the target position for upright fluoroscopy and the target position for decubitus fluoroscopy are each defined with respect to five directions of the X-axis direction, the Y-axis direction, the Z-axis direction, the α direction, and the β direction. The target positions and the allowable range shown in FIG. 19 are stored in the memory 62 as data. The controller 50 compares the detection values of the positions acquired by the detection sensors 37A to 37E with the allowable ranges centered on the target values for the five directions shown in FIG. 19. For example, in a case in which three of the positions in the five directions of the radiation source 13 are included in the allowable range for upright fluoroscopy and two of the positions are included in the allowable range for decubitus fluoroscopy, the controller 50 determines that the fluoroscopy intended by the user is upright fluoroscopy. In addition to the Y-axis direction, the Z-axis direction, the α direction, and the β direction, target values and allowable ranges of a first direction width and a second direction width of the irradiation field adjusted by the adjustment knobs 14A and 14B of the collimator device 14 may be used as determination parameters.

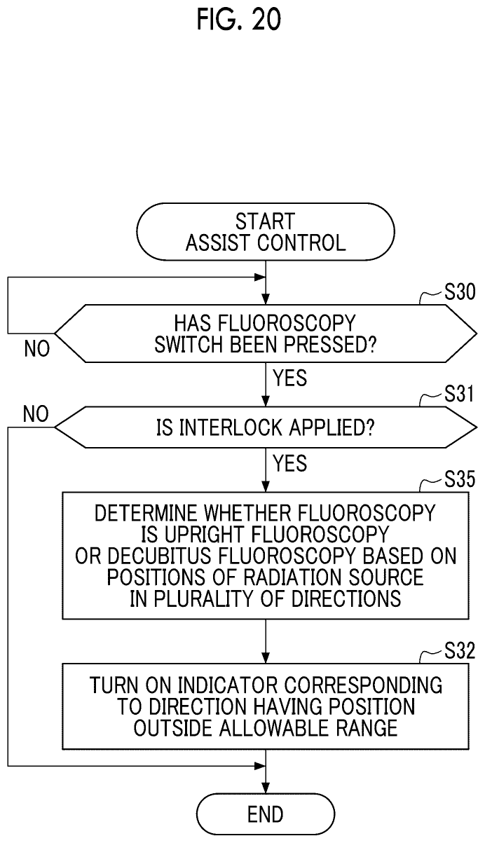

Further, after determining whether the fluoroscopy is upright fluoroscopy or decubitus fluoroscopy, the controller 50 may perform the above-mentioned assist control for the direction having a position outside the allowable range. FIG. 20 shows an assist control according to the present modification example. The assist control according to the present modification example differs from the assist control according to the first embodiment in that Step S35 is executed before Step S32 instead of Step S32. In the present modification example, in a case in which it is determined that the interlock is applied (Step S31: YES), the controller 50 determines whether the fluoroscopy intended by the user is upright fluoroscopy or decubitus fluoroscopy based on the positions of the radiation source 13 in the plurality of directions as described above (Step S35). Then, the controller 50 turns on the indicator 25 corresponding to the direction having a position outside the allowable range for the determined imaging (Step S32). For example, in a case in which three of the positions in the five directions of the radiation source 13 are included in the allowable range for upright fluoroscopy and two of the positions are included in the allowable range for decubitus fluoroscopy, the controller 50 determines that the fluoroscopy intended by the user is upright fluoroscopy and turns on the indicator 25 corresponding to the direction having a position outside the allowable range for upright fluoroscopy.

In Step S35, as described in the second embodiment, only the brake applied by the brake mechanism 36 corresponding to the direction having a position outside the allowable range may be allowed to be released.

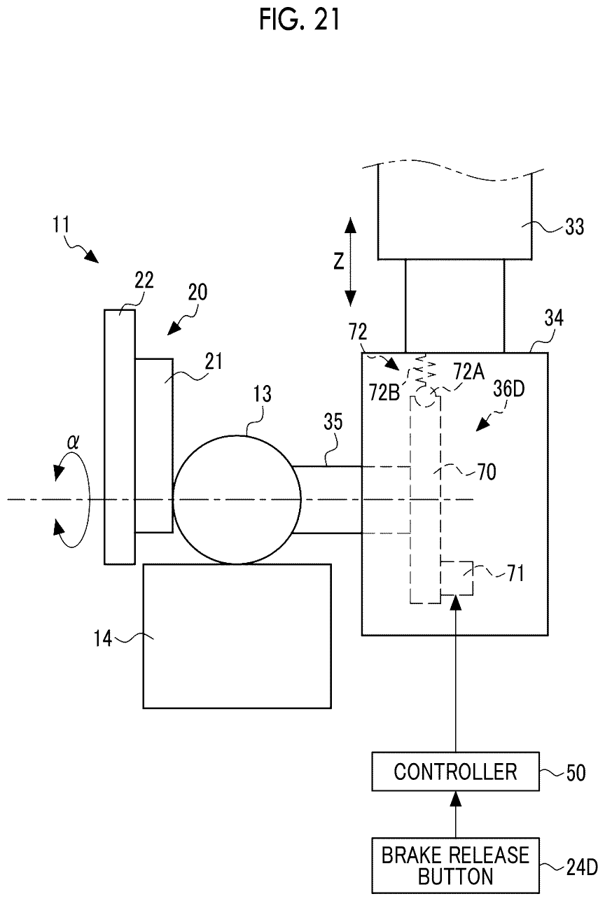

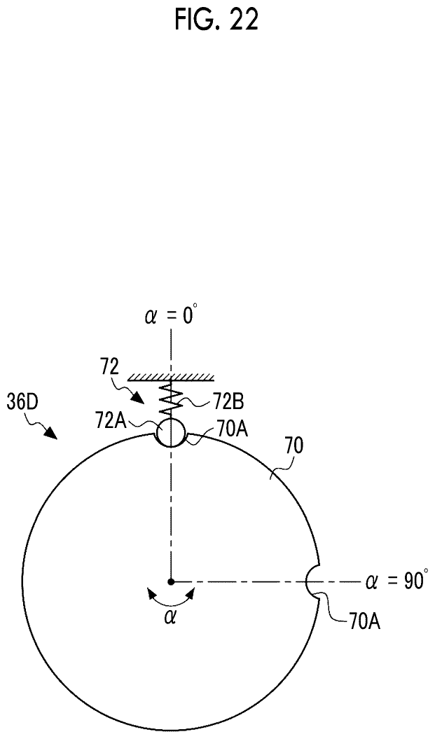

FIGS. 21 and 22 each show a modification example of the brake mechanism 36D that restricts the movement of the radiation source 13 in the α direction. FIG. 21 is a side view of the radiation irradiation device 11. FIG. 22 is a front view of the brake mechanism 36D. The brake mechanism 36D is provided in the housing of the radiation source holding portion 34.

The brake mechanism 36D includes a disk-shaped brake pad 70 and an electromagnet 71. The brake pad 70 is fixed to the rotation support portion 35. The electromagnet 71 is provided in the vicinity of the brake pad 70 and is controlled by the controller 50. The controller 50 performs on/off control of the brake mechanism 36D by controlling the current flowing through the electromagnet 71.

Further, the brake mechanism 36D is provided with a plunger 72 for positioning the position of the radiation source 13 in the α direction at a specific position. The plunger 72 is composed of a ball 72A and a spring 72B that biases the ball 72A against the outer periphery of the brake pad 70. At least one notch 70A into which the ball 72A is fitted is formed on the outer periphery of the brake pad 70. The notch 70A is provided at specific rotational positions (α=0°, 90°, and the like) at which the alignment is frequently performed. In a case in which the ball 72A is fitted into the notch 70A, the plunger 72 gives a clicking sensation to the user. With the plunger 72, the user can easily align the radiation source 13 at a specific position. In addition, the user can ascertain that the alignment has been completed by obtaining a clicking sensation.

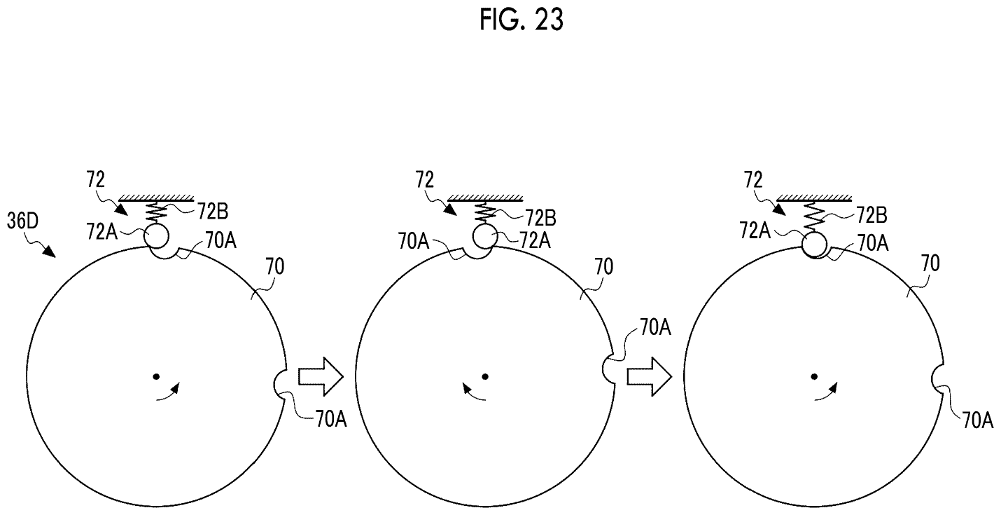

In addition, the plunger 72 has an alignment function that physically aligns the plunger 72 even in a case in which the plunger 72 is slightly misaligned from a target position during alignment, as a function different from the clicking sensation. Specifically, as shown in FIG. 23, in a case in which the ball 72A is slightly misaligned from the position where the ball 72A is fitted into the notch 70A during the alignment, the ball 72A vibrates in the α direction around the fitting position, and then is fitted into the notch 70A. In this manner, a certain amount of time is required until the position converges by the alignment function of the plunger 72. Therefore, for example, in a case in which the user aligns the radiation source 13 in the a direction while pressing the brake release button 24D and releases the pressing of the brake release button 24D before the position converges due to the alignment function of the plungers 72, the brake mechanism 36D will apply the brake at a position that is shifted from the fitting position such as α=0°. For example, in a case in which the SID is 1800 mm, even in a case in which the misregistration is 0.3°, the interlock will be applied and fluoroscopy will not be possible.

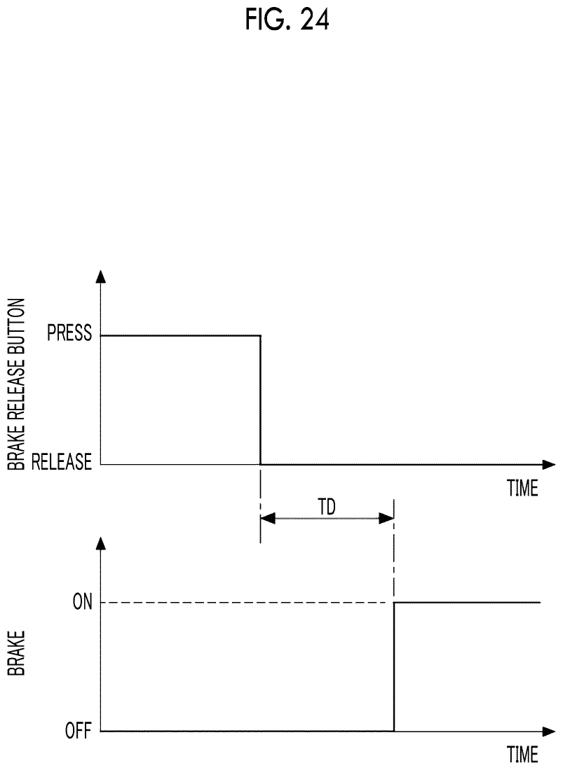

Therefore, as shown in FIG. 24, it is preferable that the controller 50 causes the brake mechanism 36D to apply the brake, after a delay time TD has elapsed from the point in time at which the controller 50 detects that the pressing of the brake release button 24D has been released. The delay time TD is a time that is sufficient for the position to converge by the alignment function of the plungers 72 and is, for example, 0.5 seconds. It is preferable that the delay time TD is provided in a case in which the assist control is performed (that is, in a case of the fluoroscopy mode). In the case of the general imaging mode, while quick imaging is required, a slight misregistration is not a problem in many cases. Therefore, it is preferable to apply the brake immediately after the release of the pressing of the brake release button 24D without providing the delay time TD.

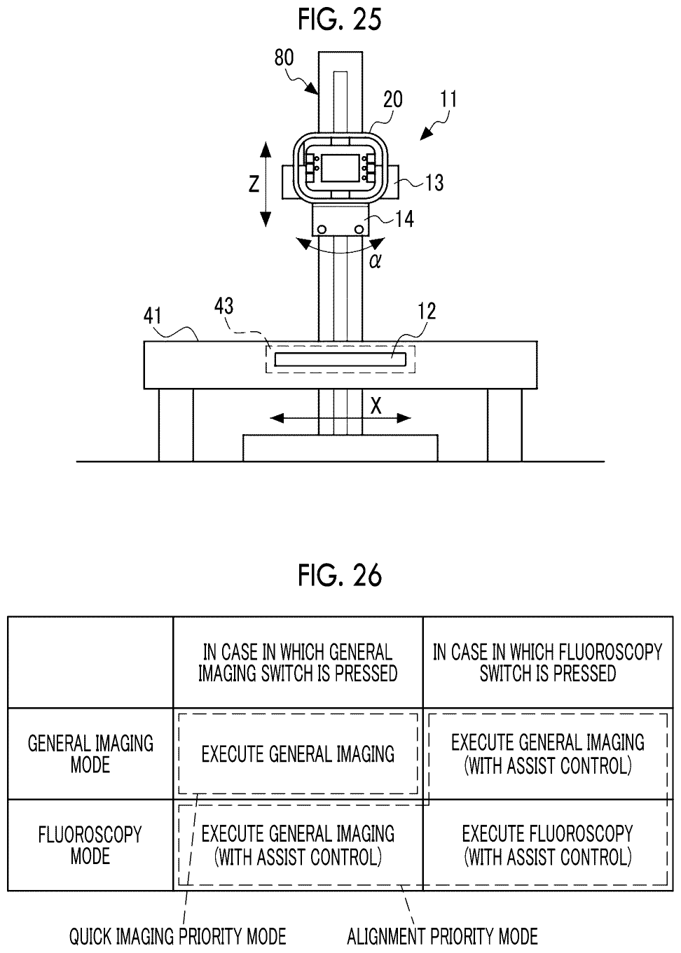

Moreover, in each of the above embodiments, the radiation source 13 is moved by the ceiling device, but the radiation source 13 may be moved by a floor device. FIG. 25 shows an example of a floor device 80. For example, the floor device 80 is used together with the decubitus imaging table 41. The floor device 80 is included in the moving device 30 described above. For example, the floor device 80 holds the radiation source 13 movably in the X-axis direction and the Z-axis direction. The floor device 80 may hold the radiation source 13 movably in the Y direction and the β direction in addition to the X-axis direction and the Z-axis direction.

That is, the moving device 30 of the present disclosure is not limited to the six directions of the X-axis direction, the Y-axis direction, the Z-axis direction, the α direction, and the β direction, and it is sufficient that the moving device 30 is configured to be capable of moving the radiation source 13 in a plurality of directions.

In addition, in the above embodiments, the plurality of brake mechanisms that restrict the movement of the radiation source 13 in the plurality of directions are provided, but it is sufficient that at least one brake mechanism is provided. That is, one brake mechanism may restrict the movement of the radiation source 13 in two or more directions.

In addition, in each of the above embodiments, the controller 50 performs an interlock control and an assist control in the case of fluoroscopy, but even in general imaging, there are cases in which strict alignment of the position of the radiation source 13 is required. For example, in a case in which the temporal change in fracture treatment is evaluated by general imaging, it is required to align the radiation source 13 at the same position every time. In addition, for example, in a case in which the head is imaged by general imaging in order to avoid the crystalline lens of the eye, strict alignment of the radiation source 13 is required. This is because any misregistration increases the risk of exposure. In this way, even in general imaging, there are cases in which it is desired to perform imaging quickly by freely moving the radiation source 13, and cases in which it is desired to perform imaging after strict alignment of the radiation source 13 even though it takes time.

In each of the above embodiments, the controller 50 executes the general imaging by pressing the general imaging switch 52 in the general imaging mode, and executes the fluoroscopy by pressing the fluoroscopy switch 53 in the fluoroscopy mode. The controller 50 may execute the general imaging in a case in which the fluoroscopy switch 53 is pressed in the general imaging mode and a case in which the general imaging switch 52 is pressed in the fluoroscopy mode.

In addition, as shown in FIG. 26, in a case in which the general imaging switch 52 is pressed in the general imaging mode, the controller 50 may determine that a “quick imaging priority mode” in which quick imaging is prioritized over alignment has been selected, and in a case in which the fluoroscopy switch 53 is pressed in the general imaging mode and a case in which the general imaging switch 52 or the fluoroscopy switch 53 is pressed in the fluoroscopy mode, the controller 50 may determine that an “alignment priority mode” in which alignment is prioritized has been selected. The controller 50 performs the above-mentioned interlock control and assist control in the alignment priority mode. In the fluoroscopy mode, since the determination is made as the “alignment priority mode” even in a case in which any one of the general imaging switch 52 or the fluoroscopy switch 53 has been pressed, the controller 50 may determine that the “alignment priority mode” has been selected in response to the switching of the imaging mode from the general imaging mode to the fluoroscopy mode as in the modification example described with reference to FIG. 18. The alignment priority mode corresponds to a “first imaging mode” according to the technology of the present disclosure. The quick imaging priority mode corresponds to a “second imaging mode” according to the technology of the present disclosure.

Therefore, the technology of the present disclosure enables selection between the quick imaging priority mode and the alignment priority mode and performs an interlock control and an assist control in a case in which the alignment priority mode is selected.

In addition, the technology of the present disclosure can be applied to a system in which an image of a subject is captured using other radiation such as γ-rays, in addition to X-rays.

In each of the embodiments and the modification examples, for example, the hardware structure of the processing unit that executes the processes, such as the controller 50, is various processors as shown below.

A CPU, a programmable logic device (PLD), a dedicated electrical circuit, and the like are included in various processors. The CPU is a general-purpose processor that executes software (programs) and functions as various processing units, as is well known. The PLD is a processor of which a circuit configuration can be changed after manufacturing, such as a field-programmable gate array (FPGA). The dedicated electrical circuit is a processor having a circuit configuration specially designed for executing specific processing, such as an application-specific integrated circuit (ASIC).

One processing unit may be configured by one of various processors, or may be configured by a combination of two or more processors of the same or different kinds (for example, a combination of a plurality of FPGAs or a combination of a CPU and an FPGA). In addition, a plurality of processing units may be configured by one processor. As an example of configuring a plurality of processing units via one processor, first, there is a form in which one processor is configured by a combination of one or more CPUs and software, and this processor functions as a plurality of processing units. Second, as represented by a system-on-chip (SoC) or the like, there is a form of using a processor for implementing the function of the entire system including a plurality of processing units with one IC chip. Thus, various processing units are configured by using one or more of the above-described various processors as hardware structures.

Furthermore, the hardware structure of these various processors is, more specifically, an electrical circuit (circuitry) in which circuit elements such as semiconductor elements are combined.

Two or more of the above embodiments and modification examples can be combined with each other as long as no contradiction occurs.

The technology of the present disclosure is not limited to the above embodiments and modification examples, and various configurations can be employed without departing from the gist of the present disclosure. Further, the technology of the present disclosure also extends to a computer-readable storage medium that non-transitorily stores the program, in addition to the program.

The following technologies can be ascertained by the above description.

### Supplementary Note 1

A radiography system comprising:

a radiation detection device configured to detect radiation to generate a radiation image;

a radiation source configured to irradiate the radiation detection device with the radiation;

a moving device capable of changing, in a plurality of directions, at least any of a translational position or a rotational position of the radiation source with respect to the radiation detection device, the moving device including a plurality of detection sensors that detect positions of the radiation source in the plurality of directions; and

a processor configured to control the radiation source,

in which the processor is configured to execute
an interlock control for applying an interlock to prohibit the irradiation with the radiation by the radiation source in a case in which, based on detection values from the plurality of detection sensors, at least one of the positions in the plurality of directions is not included in an allowable range for a target position, and
an assist control for giving a notification of or mechanically assisting in a direction in which the radiation source is to be moved in a case in which the interlock is applied.

### Supplementary Note 2

The radiography system according to Supplementary Note 1,

in which the processor is configured to:
determine which of a first imaging mode in which alignment of the radiation source with respect to the radiation detection device is prioritized and a second imaging mode in which quick imaging is prioritized over the alignment has been selected; and
perform the interlock control and the assist control in a case in which it is determined that the first imaging mode has been selected.

### Supplementary Note 3

The radiography system according to Supplementary Note 2,

in which the processor is configured to determine that the first imaging mode has been selected in a case in which a fluoroscopy switch for giving an instruction to execute fluoroscopy is pressed.

### Supplementary Note 4

The radiography system according to Supplementary Note 2 or 3,

in which the processor is configured to determine that the first imaging mode has been selected in a case in which switching is made from a general imaging mode for executing general imaging to a fluoroscopy mode for executing fluoroscopy.

### Supplementary Note 5

The radiography system according to any one of Supplementary Notes 2 to 4,

in which the processor is configured to determine that the first imaging mode has been selected in a case in which a general imaging switch for giving an instruction to execute general imaging is pressed in a fluoroscopy mode for executing fluoroscopy.

### Supplementary Note 6

The radiography system according to any one of Supplementary Notes 1 to 5, further comprising:

a plurality of indicators corresponding to the plurality of directions,

in which the processor is configured to turn on an indicator corresponding to a direction having a position outside the allowable range among the plurality of indicators in the assist control.

### Supplementary Note 7

The radiography system according to Supplementary Note 6, further comprising:

at least one brake mechanism configured to restrict movement of the radiation source in the plurality of directions; and

an operating device that is provided with a plurality of brake release buttons for releasing a brake applied by the brake mechanism,

in which the plurality of indicators are provided in a vicinity of the plurality of brake release buttons.

### Supplementary Note 8

The radiography system according to Supplementary Note 6 or 7,

in which the processor is configured to, in a case in which the indicator corresponding to the direction having the position outside the allowable range is turned on, change a lighting color in accordance with an amount of deviation between the position of the radiation source and the target position.

### Supplementary Note 9

The radiography system according to any one of Supplementary Notes 6 to 8,

in which the processor is configured to, in a case in which the indicator corresponding to the direction having the position outside the allowable range is turned on, change a blinking cycle in accordance with an amount of deviation between the position of the radiation source and the target position.

### Supplementary Note 10

The radiography system according to any one of Supplementary Notes 1 to 9,

in which the processor is configured to display, in the assist control, a device structure diagram that schematically shows the radiation source and the direction in which the radiation source is to be moved on a display.

### Supplementary Note 11

The radiography system according to any one of Supplementary Notes 1 to 6, further comprising:

at least one brake mechanism configured to restrict movement of the radiation source in the plurality of directions; and

an operating device that is provided with a plurality of brake release buttons for releasing a brake applied by the brake mechanism,

in which the processor is configured to allow only a brake corresponding to a direction having a position outside the allowable range to be released.

### Supplementary Note 12

The radiography system according to any one of Supplementary Notes 1 to 6, further comprising:

at least one brake mechanism configured to restrict movement of the radiation source in the plurality of directions; and

an operating device that is provided with a plurality of brake release buttons for releasing a brake applied by the brake mechanism,

in which the plurality of brake release buttons include a simultaneous release button for simultaneously releasing brakes corresponding to two or more of the plurality of directions, and

the processor is configured to, in a case in which the simultaneous release button is pressed, release only a brake corresponding to a direction having a position outside the allowable range.

### Supplementary Note 13

The radiography system according to Supplementary Note 11 or 12,

in which the processor is configured to control the brake mechanism whose brake has been released, and apply a load in a direction opposite to a direction toward the target position.

### Supplementary Note 14

The radiography system according to any one of Supplementary Notes 11 to 13,

in which the processor is configured to:
perform pulse duty driving on the brake mechanism whose brake has been released; and
change a duty in accordance with a distance between the position of the radiation source and the target position.

### Supplementary Note 15

The radiography system according to any one of Supplementary Notes 1 to 14,

in which the processor is configured to:
determine whether fluoroscopy intended by a user is upright fluoroscopy or decubitus fluoroscopy based on whether the positions in the plurality of directions are included more in an allowable range of a target position for upright fluoroscopy or an allowable range of a target position for decubitus fluoroscopy; and
execute the assist control for a direction having a position outside the allowable range.

### Supplementary Note 16

The radiography system according to any one of Supplementary Notes 1 to 15, further comprising:

a brake mechanism configured to restrict movement of the radiation source in a rotation direction among the plurality of directions; and

an operating device that is provided with a brake release button for releasing a brake applied by the brake mechanism,

in which the brake mechanism includes a plunger for aligning the radiation source to a specific rotational position.

### Supplementary Note 17

The radiography system according to Supplementary Note 16,

in which the processor is configured to cause the brake mechanism to apply the brake, after a delay time has elapsed from a point in time at which it is detected that pressing of the brake release button has been released.

### Supplementary Note 18

The radiography system according to any one of Supplementary Notes 1 to 17,

in which the moving device includes a ceiling device or a floor device.

What is claimed is:

1. A radiography system comprising:
a radiation detection device configured to detect radiation to generate a radiation image;
a radiation source configured to irradiate the radiation detection device with the radiation;
a moving device capable of changing, in a plurality of directions, at least any of a translational position or a rotational position of the radiation source with respect to the radiation detection device, the moving device including a plurality of detection sensors that detect positions of the radiation source in the plurality of directions; and
a processor configured to control the radiation source,
wherein the processor is configured to execute
an interlock control for applying an interlock to prohibit the irradiation with the radiation by the radiation source in a case in which, based on detection values from the plurality of detection sensors, at least one of the positions in the plurality of directions is not included in an allowable range for a target position, and
an assist control for giving a notification of or mechanically assisting in a direction in which the radiation source is to be moved in a case in which the interlock is applied.

2. The radiography system according to claim 1,
wherein the processor is configured to:
determine which of a first imaging mode in which alignment of the radiation source with respect to the radiation detection device is prioritized and a second imaging mode in which quick imaging is prioritized over the alignment has been selected; and
perform the interlock control and the assist control in a case in which it is determined that the first imaging mode has been selected.

3. The radiography system according to claim 2,
wherein the processor is configured to determine that the first imaging mode has been selected in a case in which a fluoroscopy switch for giving an instruction to execute fluoroscopy is pressed.

4. The radiography system according to claim 2,
wherein the processor is configured to determine that the first imaging mode has been selected in a case in which switching is made from a general imaging mode for executing general imaging to a fluoroscopy mode for executing fluoroscopy.

5. The radiography system according to claim 2,
wherein the processor is configured to determine that the first imaging mode has been selected in a case in which a general imaging switch for giving an instruction to execute general imaging is pressed in a fluoroscopy mode for executing fluoroscopy.

6. The radiography system according to claim 1, further comprising:
a plurality of indicators corresponding to the plurality of directions,
wherein the processor is configured to turn on an indicator corresponding to a direction having a position outside the allowable range among the plurality of indicators in the assist control.

7. The radiography system according to claim 6, further comprising:
at least one brake mechanism configured to restrict movement of the radiation source in the plurality of directions; and
an operating device that is provided with a plurality of brake release buttons for releasing a brake applied by the brake mechanism,
wherein the plurality of indicators are provided in a vicinity of the plurality of brake release buttons.

8. The radiography system according to claim 6,
wherein the processor is configured to, in a case in which the indicator corresponding to the direction having the position outside the allowable range is turned on, change a lighting color in accordance with an amount of deviation between the position of the radiation source and the target position.

9. The radiography system according to claim 6,
wherein the processor is configured to, in a case in which the indicator corresponding to the direction having the position outside the allowable range is turned on, change a blinking cycle in accordance with an amount of deviation between the position of the radiation source and the target position.

10. The radiography system according to claim 1,
wherein the processor is configured to display, in the assist control, a device structure diagram that schematically shows the radiation source and the direction in which the radiation source is to be moved on a display.

11. The radiography system according to claim 1, further comprising:
at least one brake mechanism configured to restrict movement of the radiation source in the plurality of directions; and
an operating device that is provided with a plurality of brake release buttons for releasing a brake applied by the brake mechanism,
wherein the processor is configured to allow only a brake corresponding to a direction having a position outside the allowable range to be released.

12. The radiography system according to claim 1, further comprising:
at least one brake mechanism configured to restrict movement of the radiation source in the plurality of directions; and
an operating device that is provided with a plurality of brake release buttons for releasing a brake applied by the brake mechanism,
wherein the plurality of brake release buttons include a simultaneous release button for simultaneously releasing brakes corresponding to two or more of the plurality of directions, and
the processor is configured to, in a case in which the simultaneous release button is pressed, release only a brake corresponding to a direction having a position outside the allowable range.

13. The radiography system according to claim 11,
wherein the processor is configured to control the brake mechanism whose brake has been released, and apply a load in a direction opposite to a direction toward the target position.

14. The radiography system according to claim 11,
wherein the processor is configured to:
perform pulse duty driving on the brake mechanism whose brake has been released; and
change a duty in accordance with a distance between the position of the radiation source and the target position.

15. The radiography system according to claim 1,
wherein the processor is configured to:
determine whether fluoroscopy intended by a user is upright fluoroscopy or decubitus fluoroscopy based on whether the positions in the plurality of directions are included more in an allowable range of a target position for upright fluoroscopy or an allowable range of a target position for decubitus fluoroscopy; and
execute the assist control for a direction having a position outside the allowable range.

16. The radiography system according to claim 1, further comprising:
a brake mechanism configured to restrict movement of the radiation source in a rotation direction among the plurality of directions; and
an operating device that is provided with a brake release button for releasing a brake applied by the brake mechanism,
wherein the brake mechanism includes a plunger for aligning the radiation source to a specific rotational position.

17. The radiography system according to claim 16,
wherein the processor is configured to cause the brake mechanism to apply the brake, after a delay time has elapsed from a point in time at which it is detected that pressing of the brake release button has been released.

18. The radiography system according to claim 1,
wherein the moving device includes a ceiling device or a floor device.

19. An operation method of a radiography system including:
a radiation detection device configured to detect radiation to generate a radiation image;
a radiation source configured to irradiate the radiation detection device with the radiation; and
a moving device capable of changing, in a plurality of directions, at least any of a translational position or a rotational position of the radiation source with respect to the radiation detection device, the moving device including a plurality of detection sensors that detect positions of the radiation source in the plurality of directions, the operation method comprising
executing, by a processor,
an interlock control for applying an interlock to prohibit the irradiation with the radiation by the radiation source in a case in which, based on detection values from the plurality of detection sensors, at least one of the positions in the plurality of directions is not included in an allowable range for a target position, and
an assist control for giving a notification of or mechanically assisting in a direction in which the radiation source is to be moved in a case in which the interlock is applied.

20. A non-transitory computer-readable storage medium storing an operation program for operating a radiography system including:
a radiation detection device configured to detect radiation to generate a radiation image;
a radiation source configured to irradiate the radiation detection device with the radiation; and
a moving device capable of changing, in a plurality of directions, at least any of a translational position or a rotational position of the radiation source with respect to the radiation detection device, the moving device including a plurality of detection sensors that detect positions of the radiation source in the plurality of directions, the operation program causing a processor to execute:
an interlock control for applying an interlock to prohibit the irradiation with the radiation by the radiation source in a case in which, based on detection values from the plurality of detection sensors, at least one of the positions in the plurality of directions is not included in an allowable range for a target position; and
an assist control for giving a notification of or mechanically assisting in a direction in which the radiation source is to be moved in a case in which the interlock is applied.
